(19) United States
(12) Patent Application Publication
(10) Pub. No.: US 2016/0148535 A1
(43) Pub. Date: May 26, 2016

(54) Tracking Nutritional Information about Consumed Food

(71) Applicant: ICON Health & Fitness, Inc., Logan, UT (US)

(72) Inventor: Darren C. Ashby, Richmond, UT (US)

(21) Appl. No.: 14/945,101

(22) Filed: Nov. 18, 2015

Related U.S. Application Data
(60) Provisional application No. 62/085,200, filed on Nov. 26, 2014.
(60) Provisional application No. 62/085,202, filed on Nov. 26, 2014.

(51) Int. Cl. G09B 19/00; A61B 5/145; A61B 7/02; A61B 7/00; G09B 5/02; A61B 5/00

(52) U.S. Cl. CPC G09B 19/0092; G09B 5/02; A61B 5/4866; A61B 7/023; A61B 7/008; A61B 5/4205; A61B 5/6803; A61B 5/686; A61B 5/14532; A61B 5/7278; A61B 5/0077; A61B 2562/0219; A61B 2560/0475

(57) ABSTRACT

A system for tracking nutritional information about consumed food includes a processor and memory. The memory includes programmed instructions executable by the processor to receive a first input from a first sensor indicating a swallowing and/or chewing activity, receive a second input from a second sensor indicating a physiological response of the user, and generate a nutritional value consumed at least in part based on the first input and the second input.

### RELATED APPLICATIONS

This application claims priority to U.S. Provisional Patent Application Ser. No. 62/085,200 titled “Tracking Nutritional Information about Consumed Food” and filed on 26 Nov. 2014, and U.S. Provisional Patent Application Ser. No. 62/085,202 titled “Tracking Nutritional Information about Consumed Food with a Wearable Device” and filed on 26 Nov. 2014, which applications are herein incorporated by reference for all that they disclose.

### BACKGROUND

Those trying to lose weight often track the number of calories that they consume during a day. The goal is to consume less calories than calories that are burned through exercise and daily body maintenance. Having a deficit of calories in a day is linked to weight loss. On the other hand, body builders and some athletes desire to gain muscle. Thus, they try to eat more calories than they consume during a day. The excess calories are believed to contribute to muscle gain.

To track the number of calories eaten in a day, a user will often look at labels on food packaging and determine the amount of the food that he or she can eat. If there is no calorie information listed on the food packaging, the user may search the internet or look at publications to determine or estimate the amount of calories in the food that he or she is eating.

One type of system for tracking the amount of calories in a user's food is disclosed in U.S. Patent Publication No. 2013/0273506 issued to Stephanie Melowsky. In this reference, a system and method for collecting food intake related information includes processing the information into a caloric value, and recording and reporting the value. The system includes an electronic device having a sensor, an input device, a display, processor, memory and code modules executing in the processor for implementation of the method. Information concerning the swallowing of food is collected. Weighting factors related to the caloric concentration of the food being ingested are also collected. The caloric value of the users eating is computed by the processor by combining the swallow data with weighted parameters in accordance with an algorithm. The caloric value is recorded in a user's profile and notifications can be generated based on the caloric value and a historical record of food intake information can be maintained and provided to the user via a portal such as a smart phone device or the internet. Another type of systems is described in U.S. Patent Publication No. 2011/0276312 issued to Tadmor Shalon, et al. Both of these documents are herein incorporated by reference for all that they contain.

### SUMMARY

In one aspect of the invention, a system for tracking nutritional information about consumed food includes a processor and memory.

In one aspect of the invention, the memory comprises programmed instructions executable by the processor to receive a first input from a first sensor indicating a swallowing and/or chewing activity.

In one aspect of the invention, the memory comprises programmed instructions executable by the processor to receive a second input from a second sensor indicating a physiological response of the user.

In one aspect of the invention, the memory comprises programmed instructions executable by the processor to generate a nutritional value consumed at least in part based on the first input and the second input.

In one aspect of the invention, the first sensor comprises a microphone capable of recording sounds representative of swallowing and/or chewing activity.

In one aspect of the invention, the first sensor is incorporated into eye glasses.

In one aspect of the invention, the first sensor comprises an accelerometer.

In one aspect of the invention, the first sensor is attachable to a neck of the user.

In one aspect of the invention, the programmed instructions are further executable to determine an amount of food consumed based on the first input.

In one aspect of the invention, the programmed instructions are further executable to determine a type of food based on the second input.

In one aspect of the invention, the second sensor is incorporated into an implant.

In one aspect of the invention, the second sensor comprises a non-invasive mechanism to measure a physiological characteristic indicative of the physiological response.

In one aspect of the invention, the swallowing and/or chewing activity includes a number of swallows.

In one aspect of the invention, the swallowing and/or chewing activity includes a chewing duration.

In one aspect of the invention, the programmed instructions are further executable to send a message when a nutritional goal is exceeded.

In one aspect of the invention, the programmed instructions are further executable to send nutritional information to a database.

In one aspect of the invention, the first sensor and/or second sensor are incorporated into a wearable computing device.

In one aspect of the invention, the physiological response is a glycemic response.

In one aspect of the invention, a system for tracking nutritional information about consumed food includes a processor and memory.

In one aspect of the invention, the memory comprises programmed instructions executable by the processor to receive a first input from a first sensor capable of recording swallowing and/or chewing activity.

In one aspect of the invention, the memory comprises programmed instructions executable by the processor to receive a second input from a second sensor indicating a glycemic response.

In one aspect of the invention, the memory comprises programmed instructions executable by the processor to determine an amount of food consumed based on the first input.

In one aspect of the invention, the memory comprises programmed instructions executable by the processor to determine a type of food based on the second input.

In one aspect of the invention, the memory comprises programmed instructions executable by the processor to generate a number of calories consumed at least in part based on the amount and the type of food.

In one aspect of the invention, the programmed instructions are further executable to send the number of calories to a database.

In one aspect of the invention, the first sensor and/or second sensor are incorporated into a wearable computing device.

In one aspect of the invention, the first sensor comprises an accelerometer.

In one aspect of the invention, a system for tracking nutritional information about consumed food includes a processor and memory.

In one aspect of the invention, the memory comprises programmed instructions executable by the processor to receive a first input from a first sensor capable of recording swallowing and/or chewing activity.

In one aspect of the invention, the memory comprises programmed instructions executable by the processor to receive a second input from a second sensor indicating a glycemic response.

In one aspect of the invention, the memory comprises programmed instructions executable by the processor to determine an amount of food consumed based on the first input.

In one aspect of the invention, the memory comprises programmed instructions executable by the processor to determine a type of food based on the second input.

In one aspect of the invention, the memory comprises programmed instructions executable by the processor to determine a generate of calories consumed at least in part based on the amount and the type of food.

In one aspect of the invention, the memory comprises programmed instructions executable by the processor to send the number of calories to a database.

In one aspect of the invention, the first sensor and/or the second sensor are incorporated into a wearable computing device.

Any of the aspects of the invention detailed above may be combined with any other aspect of the invention detailed herein.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a perspective view of an example of a system for tracking a consumed amount of calories in accordance with the present disclosure.

FIG. 2 illustrates a perspective view of an example of a tracking system in accordance with the present disclosure.

FIG. 3 illustrates a block diagram of an example of a mobile device in communication with sensors for tracking an amount of calories consumed in accordance with the present disclosure.

FIG. 4 illustrates a perspective view of an example of a system for tracking a consumed amount of calories in accordance with the present disclosure.

Throughout the drawings, identical reference numbers designate similar, but not necessarily identical, elements.

### DETAILED DESCRIPTION

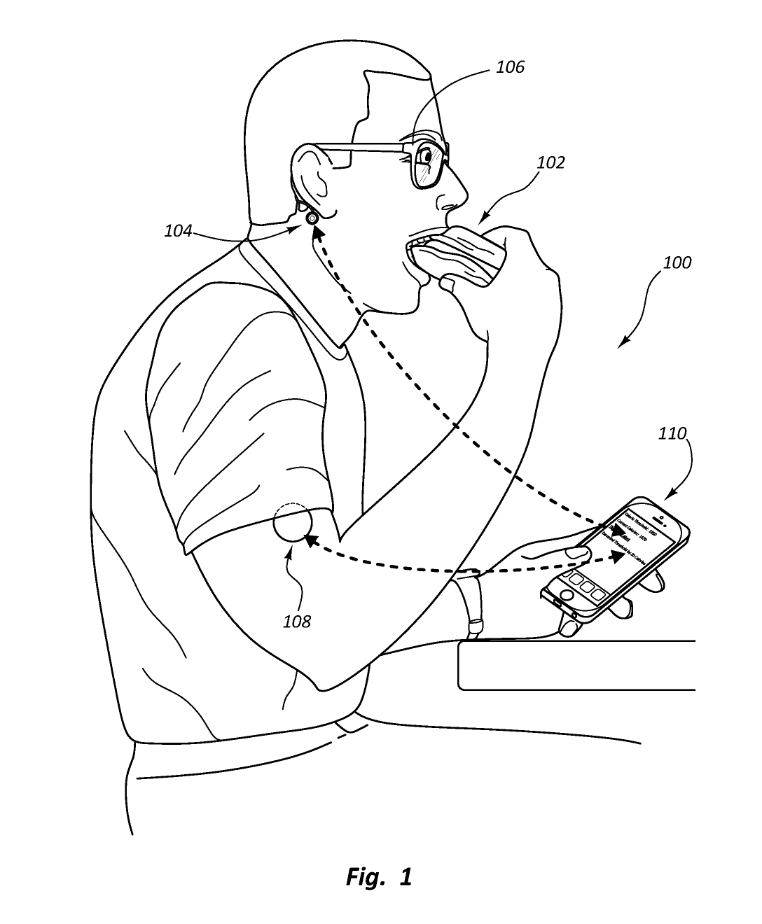

Particularly, with reference to the figures, FIG. 1 illustrates a perspective view of an example of a tracking system 100 for tracking a consumed amount of calories. In this example, a user is consuming an amount of calories by eating food 102. As the user eats, a first sensor 104 attached to the user's eye wear 106 picks up swallowing and/or chewing sounds/motions, which help to determine a volume of food that the user is eating. A second sensor 108 is attached to the user's skin, which measures a physiological response to the food being consumed by the user, such as a glycemic response or another type of response. Both the first and second sensors 104, 108 send a measured output to a mobile device 110 carried by the user. The combined outputs from the first and second sensors 104, 108 can be used to determine the amount of calories being consumed by the user.

The first sensor 104 may be a microphone, an accelerometer, another type of sensor or combinations thereof. The first sensor 104 may be positioned proximate the jawbone, the mouth, the throat, the ear, another portion of the user within in a region capable of picking up swallowing and/or chewing sounds and/or movements. The first sensor 104 may be positioned by the eye wear 106, a hat, a scarf, jewelry, a wearable device, an adhesive, another mechanism or combinations thereof.

The bones of the user's face, such as the jawbone and other bones, may conduct low frequency sound waves generated from chewing that can be picked up by a microphone proximate the user's ear. The amount of time that food is chewed may reveal characteristics about the food, such as the amount of food, the type of food, the consistency of food, other types of food characteristics or combinations thereof. In some examples, the first sensors records the amount of time that the user chews an amount of food. In such an example, the duration of time may be the sole factor used to determine the volume of food. In other examples, the types of sounds generated during chewing may be used to determine the volume of food. For example, frequency patterns that represent liquid food, soft food, brittle food, chewy food, or other types of food characteristics may be used as a factor to determine the amount of food. In one such example, if sounds are detected that indicate that the food has a chewy consistency, the calculated amount of food may be adjusted downward to reflect that the type of food may need more chews than other types of food. In the same example, soft food may be broken down from chewing with relatively less chewing than the food with the chewy consistency. As a result, detected food types may be associated with chew to volume ratios to more accurately determine the volume of food consumed by the user.

Alternatively, or in combination with recording the chewing sounds, the first sensor may record swallowing movements. In some examples, the tracking system 100 may have an assumption that each swallow of food has a consistent volume. In other examples, the number of swallows is just one among multiple factors used to determine the volume of food. In some cases, the time duration between swallows may be used as a factor to determine the volume of food. For example, a second swallow that occurs immediately after a first swallow may reflect that the first and/or the second swallow included a smaller volume of food.

The number of swallows may be recorded with a microphone of the first sensor 104. Thus, sounds that are generated through swallowing may be detected during each swallow and may be recorded. In other examples, time periods between chewing activity may also counted as swallows. For example, if chewing activity is detected and the chewing activity stops for a time before the chewing activity resumes, such a pauses in chewing activity may be counted as a swallow. In circumstances where the first sensor is configured to detect just chewing sounds, the pauses in chewing activity may represent the time that swallowing occurs or may represent that a new batch of food has replaced a previous volume of food in the mouth.

The first sensor may include an accelerometer. The accelerometer may detect movements that represent chewing and/or swallowing. For example, during chewing an accelerometer in contact with the user's jaw may detect the jaw's movement. However, the amount of tension on the user's skin may also be alternate between higher and lower amounts of tension as the jawbone moves. The varying amounts of tension may cause the skin around the ears, neck, throat, jaw and other locations of the user's head to move during chewing. The accelerometer may be positioned to detect any of these movements. Further, the user's muscles may flex and relax during chewing, and such muscle movement may also be detected by the accelerometer.

In some examples, just chewing is detected with a microphone. In other examples, just swallowing is detected with a microphone. In other examples, the first sensor includes just a microphone to detect both chewing and swallowing. In other examples, just chewing is detected with an accelerometer. In yet other examples, just swallowing is detected with an accelerometer. In further examples, the first sensor includes just an accelerometer to detect both chewing and swallowing.

The first sensor may have a processor and logic to interpret the recorded sounds and/or movements. In other situations, the first sensor may send the recordings to another device to interpret the recordings. In some examples, the first sensor may process at least a portion of the recordings to be sent to the mobile device to reduce bandwidth. For example, the first sensor may compress data, filter data or otherwise modify the data. In other examples, the first sensors includes minimal logic to reduce the amount of power needed to operate the sensor. In some examples, a battery may be fixed to the eye wear 106 or other device holding the first sensor 104. In other examples, the battery is incorporated directly into the first sensor. Further, the sensor may be powered by converting movement and/or heat of the user into useable energy.

A processor, whether located in the first sensor or in a remote device, may interpret the first sensor's recordings. In the example of FIG. 1, the processor is located in the mobile device 110. The processor may be executed by programmed instructions to determine characteristics of the recordings, such as distinguishing between the chewing and swallowing, the number of swallows, the number of chews, the time duration of chewing, the type of food being chewed, other types of characteristics or combinations thereof.

The second sensor 108 may be attached to any appropriate location of the user to measure a glycemic response to the food in the user's body. Thus, the second sensor 108 may be positioned to come into contact with the user's blood or be capable of measuring an secondary effect of the response that corresponds to a condition of the user's blood. In some examples, the second sensor 108 is implanted into the user to come into direct contact with the user's blood. In other examples, the second sensor is in direct contact with the user's skin.

In some examples, the physiological response measured by the second sensor is a glycemic response. Such a response may be measured based on the glycemic index, the glycemic load or another parameter. Foods with carbohydrates that break down quickly during digestion and release glucose rapidly into the user's blood have a higher glycemic response. On the other hand, foods with carbohydrates that break down more slowly will release glucose more gradually into the bloodstream. These types of food have lower glycemic responses. Those food with lower glycemic responses tend to have more consistent blood glucose readings after meals. On the other hand, foods with higher glycemic responses tend to cause a more rapid rise in blood glucose levels after a meal.

A glycemic index is a number associated with food types that indicates the foods effect on a person's blood sugar level. The number often ranges between fifty and hundred where hundred represents pure glucose. The glycemic index represents the total rise in a person's blood sugar level following consumption of the food. The rate at which the blood sugar rises can be influenced by a number of other factors, such as the quantity of food, the amount of fat, protein, fiber and other substances in the food.

The person's blood glucose level may be measured by measuring constituents of the user's blood, interstitial fluid, body fluid, other types of fluids, other types of tissues or combinations thereof. In some examples, the second sensor 108 is implanted into the user's body to provide direct contact to the user's blood or other body fluid/tissue. In other examples, non-invasive blood glucose monitoring systems may be used. For example, the second sensor may include near infrared detection, ultrasound spectroscopy, dielectric spectroscopy, fluorescent glucose biosensors, other types of techniques or combinations thereof.

The glycemic response may be used to determine the type of food that was consumed by the user. As the user eats the food, the food volume is recorded. As the physiological response of the food is exhibited, the food volumes may be associated with the food type identified by the physiological response. The food type and food volume may be combined to determine the number of calories that the person consumed.

While the examples above have been described as using a glycemic response to determine the food type, any appropriate physiological response may be used. For example, an insulin response may be used to determine the food type. In other examples, thermal responses, hormone responses, leptin responses, cholesterol responses, oxygen responses, enzyme responses, other types of physiological responses or combinations thereof may be measured by the second sensor 108 and used to determine a food type.

In some examples, the first and second sensors 104, 108 are calibrated to be specific for the user as mouth sizes and physiological responses vary by the person. For example, the chewing sensors may be calibrated based on the amount of fluid that the user can retain in his or her mouth and squirt into a measuring cup. However, other mechanisms for determine the user's mouth size may be used in accordance with the principles described in the present disclosure. Further, the second sensor may be calibrated by having the user eat a predetermined amount of a predetermined type of food (i.e. a teaspoon of sugar) to measure the actual glycemic response of a known quantity of a known food. Additionally, the calibration procedure may involve having the user ingest predetermined amounts of different types of food to fine tune the calibration.

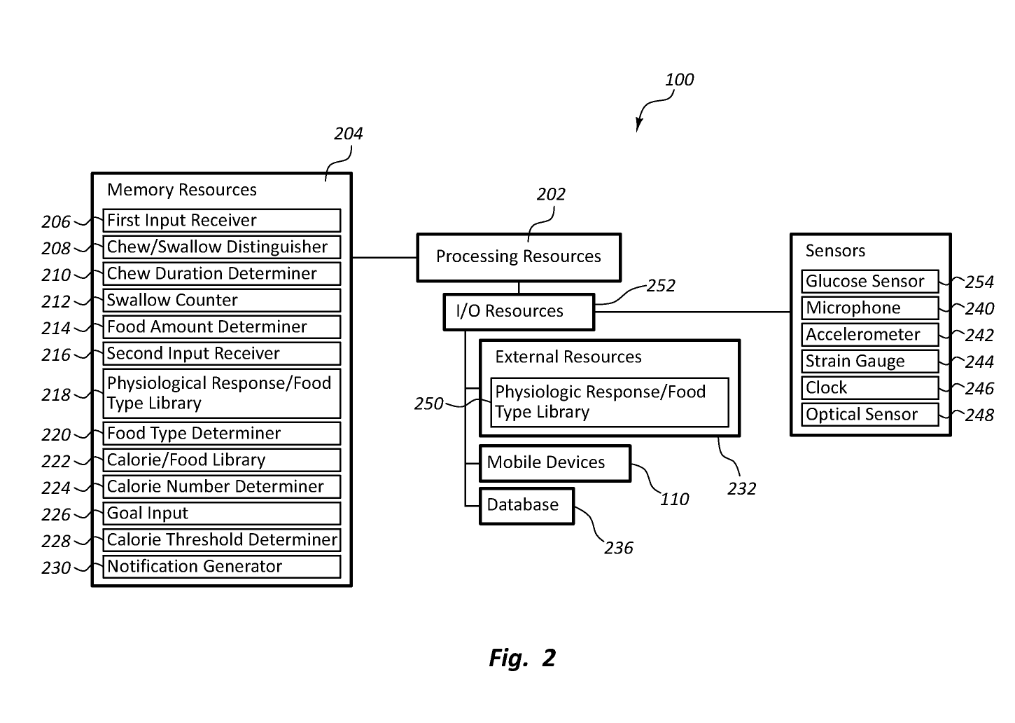

FIG. 2 illustrates a perspective view of an example of a tracking system 100 in accordance with the present disclosure. The tracking system 100 may include a combination of hardware and programmed instructions for executing the functions of the tracking system 100. In this example, the tracking system 100 includes processing resources 202 that are in communication with memory resources 204. Processing resources 202 include at least one processor and other resources used to process the programmed instructions. The memory resources 204 represent generally any memory capable of storing data such as programmed instructions or data structures used by the tracking system 100. The programmed instructions and data structures shown stored in the memory resources 204 include a first input receiver 206, a chew/swallow distinguisher 208, a chew duration determiner 210, a swallow counter 212, a food amount determiner 214, a second input receiver 216, a physiological response/food type library 218, a food type determiner 220, a calorie/food library 222, a calorie number determiner 224, a goal input 226, a calories threshold determiner 228 and a notification generator 230.

The processing resources 202 may be in communication with a remote device that stores the user information, eating history, workout history, external resources 232, databases 236 or combinations thereof. Such a remote device may be a mobile device 110, a cloud based device, a computing device, another type of device or combinations thereof. In some examples, the system communicates with the remote device through a mobile device 110 which relays communications between the tracking system 100 and the remote device. In other examples, the mobile device 110 has access to information about the user. In some cases, the remote device collects information about the user throughout the day, such as tracking calories, exercise, activity level, sleep, other types of information or combination thereof. In one such example, a treadmill used by the user may send information to the remote device indicating how long the user exercised, the number of calories burned by the user, the average heart rate of the user during the workout, other types of information about the workout or combinations thereof.

The remote device may execute a program that can provide useful information to the tracking system 100. An example of a program that may be compatible with the principles described herein includes the iFit program which is available through www.ifit.com and administered through ICON Health and Fitness, Inc. located in Logan,

Utah, U.S.A. An example of a program that may be compatible with the principles described in this disclosure are described in U.S. Pat. No. 7,980,996 issued to Paul Hickman. U.S. Pat. No. 7,980,996 is herein incorporated by reference for all that it discloses. In some examples, the user information accessible through the remote device includes the user's age, gender, body composition, height, weight, health conditions, other types of information or combinations thereof.

The processing resources 202, memory resources 204 and remote devices may communicate over any appropriate network and/or protocol through the input/output resources 252. In some examples, the input/output resources 252 includes a transceiver for wired and/or wireless communications. For example, these devices may be capable of communicating using the ZigBee protocol, Z-Wave protocol, BlueTooth protocol, Wi-Fi protocol, Global System for Mobile Communications (GSM) standard, another standard or combinations thereof. In other examples, the user can directly input some information into the tracking system 100 through a digital input/output mechanism, a mechanical input/output mechanism, another type of mechanism or combinations thereof.

The memory resources 204 include a computer readable storage medium that contains computer readable program code to cause tasks to be executed by the processing resources 202. The computer readable storage medium may be a tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory or combinations thereof.

The first input receiver 206 represents programmed instructions that, when executed, cause the processing resources 202 to receive input from the first sensor 104. Such inputs may include movements that represent chewing and/or swallowing. Also, the inputs may include sounds that represent the chewing and/or swallowing. In some cases, the inputs reflect just chewing or just swallowing. The first sensor 104 may include a microphone 240, an accelerometer 242, a magnetic device, a strain gauge 244, a clock 246, an optical sensor 248, another type of sensor or combinations thereof. For example, the strain gauge may be used to determine the movement of the user's skin. Further, the optical sensor may include a camera that detects the position of the user's jawbone, muscles, skin or other types features of the user's head. Such a camera may operate in the visual light spectrum. In other examples, the camera may operate in the infrared light spectrum.

In some examples, the chew/swallow distinguisher 208 represents programmed instructions that, when executed, cause the processing resources 202 to distinguish between inputs that represent chewing and inputs that represent swallowing. In some examples, the frequency detected by the first sensor 104 are received. The frequencies may be analyzed for patterns. Some of the patterns may exhibit characteristics of chewing while other patterns exhibit characteristics of swallowing. Further, filters may be used to remove those ranges of frequencies that usually do not represent swallowing or chewing. For example, speaking by the user or those nearby the user may also be picked up by a microphone used to detect chewing and/or swallowing, but the frequencies generated through speaking may be frequencies that do not usually depict chewing or swallowing and therefore, such frequencies are removed.

The chew duration determiner 210 represents programmed instructions that, when executed, cause the processing resources 202 to determine the time duration that food is being chewed. Such a time duration may provide an indicator as to the type of food, the amount of food in the user's mouth, other types of information or combinations thereof. The swallow counter 212 represents programmed instructions that, when executed, cause the processing resources 202 to track a number of swallows executed by the user. The swallow count may be used to determine information about the type of food, the amount of food or other characteristics of the food being ingested by the user.

The food amount determiner 214 represents programmed instructions that, when executed, cause the processing resources 202 to determine the amount of food consumed by the user based on the chew duration, the swallow count, the user's mouth volume, other factors or combinations thereof. While this example has been described with reference to specific factors for determining the amount of food, any appropriate factors for determining the amount of food may be used.

The second input receiver 216 represents programmed instructions that, when executed, cause the processing resources 202 to receive input from the second sensor 108. The input from the second sensor 108 may include information reflective of a physiological response of the user based on the type of food consumed by the user. For example, the second input may reflect a glycemic response, an insulin response, a thermal response, an oxygen response, a hormone response, an alertness response, another types of response or combinations thereof. Any appropriate type of sensor may be the second sensor 108. For example, the second sensor 108 may be a glucose sensor 254, an insulin sensor, a thermometer, another type of sensor or combinations thereof.

The physiological response/food type library 218 contains associations between the physiological response detected by the second sensor and the food type. For example, the physiological response/food type library 218 may track at least portions of the glycemic index. In such an example, if the user's glycemic response correlates with a response in the glycemic index, the food type determiner 220 may determine that the type of food that caused that glycemic response is the type of food associated with that level of response in the glycemic index. In some examples, the memory resources 204 contain a physiological response/food type library 218. However, in other examples, a physiological response/food type library 250 is accessible through the input/output resources 252. In some examples, a physiological response/food type library may be accessible through the input/output resources 252 and the memory resources 204.

The food type determiner 220 represents programmed instructions that, when executed, cause the processing resources 202 to determine the type of food. In some examples, the food type determiner 220 relies solely on the input from the second sensor to determine the food type. However, in other examples, the food type determiner 220 considers additional factors. In such an example, the chewing information, the swallowing information or other types of information may be weighed as factors for determining the food type. In yet other examples, a user eating history may be used as a factor. In such a situation if the food type determiner 220 identifies that the user often eats a particular type of food that has a similar response number on the glycemic index with another type of food, the food type determiner 220 may conclude that the more commonly eaten food of the user is the food that is currently being consumed by the user. In other examples, the geography of the user may also be used as a factor for determining what the user is eating. For example, if a location finding program on the user's smartphone indicates that the user is standing in an ice cream shop, the food type determiner 220 may place a greater weight to those foods that are available at such a location.

The food type determiner 220 may also determine that multiple types of foods are being consumed by the user. For example, the user may eat meat during a first bite and rice during a second bite. Thus, the first input and the second input may be analyzed such that the food type determiner 220 makes an independent determination about the food type for each bite. In yet other examples, the user may eat two different types of food in a single bite. In such an example, the food type determiner 220 may determine that based on the volume of food being consumed that the physiological response is being affected by multiple types of foods. In one such situation, if the user eats a first type of food with a low glycemic response during a first bite and a second type of food with a high glycemic response during a second bite, the food type determiner 220 may determine the first type of food and the second type of food with a high degree of confidence. Once these types of foods have been identified, the food type determiner 220 may determine that these types of foods are part of the meal being consumed by the user. As a result, the food type determiner 220 may look for evidence of either type of food during subsequent bites. Further, if the food type determiner 220 is unable to determine a food type with confidence, the food type determiner 220 may look at the other types of foods in other bites that were determined with a higher amounts of confidence. For example, if the first bite is determined with a low amount of confidence, but the second bite is determined to be chicken with a high confidence and the third bite is determined to be rice with a high confidence, the food type determiner 200 may consider whether the first bite contained a combination of rice and chicken.

While the examples above have been described to include specific factors for determining a food type, any appropriate factors may be used to determine the food types. A non-exhaustive list of factors that may be used to determine the food type include chew duration, swallow count, mouth volume, physiological response, physiological response time, user's eating history, user's food preferences, user's location, other types of food determined to be part of the user's meal, other factors or combinations thereof.

The calorie number determiner 224 represents programmed instructions that, when executed, cause the processing resources 202 to determine the number of calories that the user is consuming The calorie number determiner 224 may consult with the calorie/food library 222, which associates a specific number of calories per volume of a food type. The calorie number determiner 224 may determine a number of calories per bite. In other examples, the calorie number determiner 224 determines a single overall calorie count for an entire meal or time period, such as a day. In some examples, the calorie number determiner 224 maintains a running calorie total for a predetermined time period. In other examples, the calorie number determiner 224 tracks the number of calories consumed by the user for multiple time periods. The calorie number determiner 224 may track calories for a specific meal, a day, a week, another time period or combinations thereof.

The goal input 226 represents programmed instructions that, when executed, cause the processing resources 202 to allow a user to input a food/nutritional related goal, such as a calorie goal, into the tracking system 100. The calorie threshold determiner 228 represents programmed instructions that, when executed, cause the processing resources 202 to determine whether the calorie goal has been exceeded. The notification generator 230 represents programmed instructions that, when executed, cause the processing resources 202 to generate a notification to the user about the status of the goal. For example, the notification generator 230 may send a notification in response to the user exceeding his or her calorie goal. In other examples, the notification generator 230 may send a notification to the user indicating that the user is approaching his or her calorie goal. In yet other examples, the notification generator 230 may indicate whether the pace that the user is on will cause the user to exceed or fall short of his or her calorie goal.

The notification generator 230 may send notifications to the user through any appropriate mechanism. For example, the notification generator 230 may cause an email, a text message, another type of written message or combinations thereof to be sent to the user. In other examples, the notification generator 230 may cause an audible message to be spoken to the user. In yet other examples, the notification generator 230 may cause a vibration or another type of haptic event to occur to indicate to the user a notification related to the user's goal.

While the examples above have been described with reference to determining a number of calories being consumed by the user, the principles above may be applied to determining other types of information about the food being consumed by the user. For example, the principles described in the present disclosure may be used to determine the amounts of protein, fat, salt, vitamins, other types constituents or combinations thereof. Such nutritional information may be reported to the user through the same or similar mechanisms used to report the calorie information to the user. Such nutritional information may be ascertained through appropriate libraries that associate the food constituents with the food type per food volume. Further, the user may set goals pertaining to these other nutritional aspects as well. For example, the user may set goals to stay under a certain amount of salt or to consume at least a specific number of grams of protein in a day. The notification generator 230 may notify the user accordingly for such salt intake and protein consumption goals as described above.

Further, the memory resources 204 may be part of an installation package. In response to installing the installation package, the programmed instructions of the memory resources 204 may be downloaded from the installation package's source, such as a portable medium, a server, a remote network location, another location or combinations thereof. Portable memory media that are compatible with the principles described herein include DVDs, CDs, flash memory, portable disks, magnetic disks, optical disks, other forms of portable memory or combinations thereof. In other examples, the program instructions are already installed. Here, the memory resources 204 can include integrated memory such as a hard drive, a solid state hard drive or the like.

In some examples, the processing resources 202 and the memory resources 204 are located within the first sensor 104, the second sensor 108, a mobile device 110, an external device, another type of device or combinations thereof. The memory resources 204 may be part of any of these device's main memory, caches, registers, non-volatile memory or elsewhere in their memory hierarchy. Alternatively, the memory resources 204 may be in communication with the processing resources 202 over a network. Further, data structures, such as libraries or databases containing user and/or workout information, may be accessed from a remote location over a network connection while the programmed instructions are located locally. Thus, the tracking system 100 may be implemented with the first sensor 104, the second sensor 108, the mobile device 110, a phone, an electronic tablet, a wearable computing device, a head mounted device, a server, a collection of servers, a networked device, a watch or combinations thereof. Such an implementation may occur through input/output mechanisms, such as push buttons, touch screen buttons, voice commands, dials, levers, other types of input/output mechanisms or combinations thereof. Any appropriate type of wearable device may include, but are not limited to glasses, arm bands, leg bands, torso bands, head bands, chest straps, wrist watches, belts, earrings, nose rings, other types of rings, necklaces, garment integrated devices, other types of devices or combinations thereof.

The tracking system 100 of FIG. 2 may be part of a general purpose computer. However, in alternative examples, the tracking system 100 is part of an application specific integrated circuit.

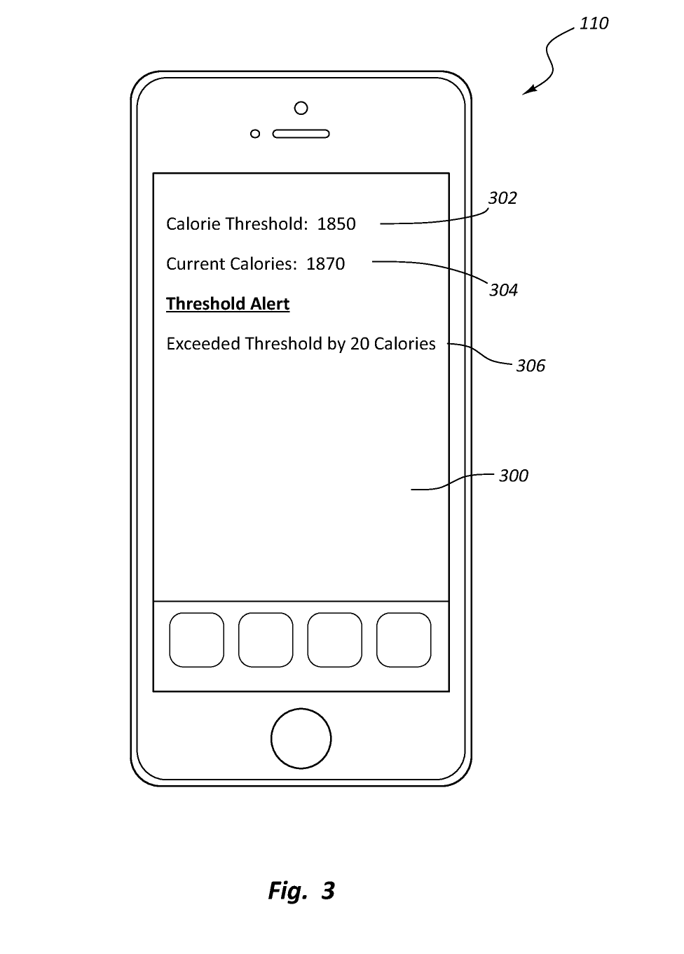

FIG. 3 illustrates a block diagram of an example of a mobile device 110 in communication with sensors for tracking an amount of calories consumed in accordance with the present disclosure. In this example, the mobile device 110 is a phone carried by the user. However, any appropriate type of mobile device may be used in accordance with the principles described in the present disclosure. For example, the mobile device 110 may include an electronic tablet, a personal digital device, a laptop, a digital device, another type of device or combinations thereof. Further, while this example is described with reference to a mobile device 110, any appropriate type of device may be used to communicate the status of the user's nutritional goals.

In the illustrated example, the mobile device 110 includes a display 300 that depicts the user's calorie goal 302 and the running total 304 of calories consumed by the user. The user may input his or her goal into the mobile device 110 or another device in communication with the tracking system 100. The user may use any appropriate mechanism for inputting the goal, such as a speech command, manual command or another type of command. The manual commands may include using buttons, touch screens, levers, sliders, dials, other types of input mechanisms or combinations thereof.

The running total 304 of calories may be determined by the tracking system 100. The tracking system 100 may update the number of calories in response to determining an additional amount of calories is consumed. In some examples, the physiological response is delayed from the moment that the user eats his or her food. As a result, the amount of calories consumed in the running total 304 may be updated after the meal has concluded. In some examples, the physiological response is manifested shortly after a meal such that the mobile device 110 may display to the user an accurate calorie count within minutes of consuming the food. In other examples, the calorie amount is updated after several hours because the physiological response takes that long to occur. In some examples where the physiological response takes a significant time to complete, the tracking system 100 may estimate the amount of calories based on an initial characteristics of the physiological response and refine the amount of calories after the physiological response is finished.

In the illustrated example, the display 300 includes a notification message 306 that the user has exceed his or her calorie goal by twenty calories. In some examples, the notification message 306 indicates the amount of calories exceeded, while in other examples, the notification message merely indicates that the goal has been exceeded without identifying the specific number of calories. In some cases, the notification message is displayed just in response to the user exceeding his or her goal. In other examples, other notification messages may be displayed prior to the calorie goal being exceeded. While the above examples have been described with a specific look and feel, any appropriate look and feel may be used to communicate to the user information about his or her food consumption, goals, other information or combinations thereof.

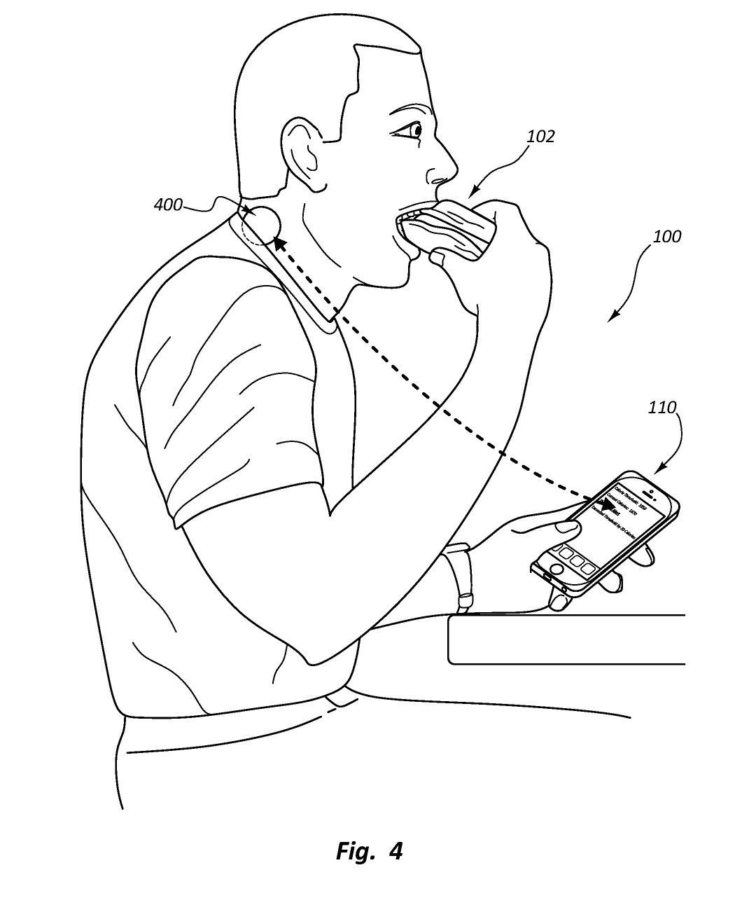

FIG. 4 illustrates a perspective view of an example of a system for tracking a consumed amount of calories in accordance with the present disclosure. In this example, the first and second sensors 104, 108 are integrated into a single patch 400 adhered to the back of the user's neck. In this example, the patch may include a strain gauge that senses the movement of the user's skin based on chewing and swallowing activities. The patch may also include a mechanism that puts the second sensor into contact with the user's blood, an interstitial fluid of the user or otherwise provides a way to where the second sensor can continuously monitor the user for the physiological response to his or her food.

The first and second sensors 104, 108 may send their inputs to the mobile device 110 to display the number of calories or other nutritional information about the food consumed by the user. In some examples, the processing and interpreting of the first and second inputs may be performed at the patch 400, while in other examples, such processing and interpreting occurs at the mobile device 110 or another remote device.

While the examples above have been described with reference to a specific first sensor, it is understood that the first sensor may be a single sensor or a group of sensors that measure chewing and/or swallowing activity. Likewise, it is understood that the second sensor may be a single sensor or a group of sensors that measure a physiological response of the user to consumed food.

Also, while the examples about have been described above with reference to determine a specific food type, it is understood that the determination of a food type may include determining that the food belongs to a specific category of food. For example, based on the first and second inputs, the system may determine that the consumed food is a food containing a high amount of carbohydrates and categorize the food as being a “high carbohydrate” type of food. In some examples, the system may not attempt to distinguish between certain types of food, especially where the distinction between food types may yield negligible differences. For example, it may not be significant for the system to distinguish between rice and pastas. Likewise, distinguishing between different types of poultry may not yield significant differences. As such, the system may broadly determine the food type without identifying the specific scientific name of the food, the food's brand or other identifiers. However, in some examples, the system may make such distinctions and narrowly identify each food type.

### INDUSTRIAL APPLICABILITY

In general, the invention disclosed herein may provide the user with a convenient system for counting the number of calories that the user consumes within a time period. This may be accomplished by placing sensors on the user that can determine the amount of food that the user is consuming as well as identify the type of food that the user in consuming By combining the volume of food with the type of food, the system can ascertain through look-up libraries the number of calories that the user has consumed. In some examples, other nutritional information can also be displayed to the user.

The user may set a goal to consume more or less than a specific number of calories. Such a goal may be inputted into the system through any appropriate input mechanism. As the user consumes food, status notifications may be sent to the user on a regular basis or in response to exceeding the goals.

The food volume may be determined based on the amount of chewing and/or swallowing that occurs as the user digests the food. In some situations, the user's mouth size is determined so that the chewing and swallowing activity is calibrated specific to the user. Likewise, the system may also be calibrated to match the user's specific physiological responses to food. In some cases, multiple physiological responses may be monitored by the second sensor or groups of second sensors. In such cases, the system may use at least one of these physiological responses to determine the food type.

Either the first and/or second sensors may be positioned through any appropriate mechanism. For example, these sensors may be positioned with eye wear, adhesives, hats, jewelry, clothing, head gear, other mechanism or combinations thereof. In some examples, the first and/or second sensor is included on an implant. The mechanism used to position the first and second sensor may free the user from hassling with the sensors while eating.

The calorie number, the volume of food, the type of food, other nutritional data or combinations thereof may be sent to remote database for storage. Such remote storage may be accessible to the user over a network, such as the internet. The user may access the records of his or her eating history, determine eating patterns and habits and make adjustments. In some situations, this nutritional information may be stored in a database or be accessible to a user profile of an exercise program, such as can be found at www.ifit.com as described above. In some examples, this nutritional information may be made public at the user's request or be made viewable to certain people. Such individuals may give the user advise about improving eating habits. In other examples, the user may compete with others to have lower amounts of calories within a time period or to achieve a different type of nutritional goal.

What is claimed is:

1. A system for tracking nutritional information about consumed food, comprising:
a processor and memory, the memory comprising programmed instructions executable by the processor to:
receive a first input from a first sensor indicating a swallowing and/or chewing activity;
receive a second input from a second sensor indicating a physiological response of a user; and
generate a nutritional value consumed at least in part based on the first input and the second input.

2. The system of claim 1, wherein the first sensor comprises a microphone capable of recording sounds representative of swallowing and/or chewing activity.

3. The system of claim 1, wherein the first sensor is incorporated into eye glasses.

4. The system of claim 1, wherein the first sensor comprises an accelerometer.

5. The system of claim 1, wherein the first sensor is attachable to a neck of the user.

6. The system of claim 1, wherein the programmed instructions are further executable to determine an amount of food consumed based on the first input.

7. The system of claim 1, wherein the programmed instructions are further executable to determine a type of food based on the second input.

8. The system of claim 1, wherein the second sensor is incorporated into an implant.

9. The system of claim 1, wherein the second sensor comprises a non-invasive mechanism to measure a physiological characteristic indicative of the physiological response.

10. The system of claim 1, wherein the swallowing and/or chewing activity includes a number of swallows.

11. The system of claim 1, wherein the swallowing and/or chewing activity includes a chewing duration.

12. The system of claim 1, wherein the programmed instructions are further executable to send a message when a nutritional goal is exceeded.

13. The system of claim 1, wherein the programmed instructions are further executable to send the nutritional information to a database.

14. The system of claim 1, wherein the first sensor and/or the second sensor are incorporated into a wearable computing device.

15. The system of claim 1, wherein the physiological response is a glycemic response.

16. A system for tracking nutritional information about consumed food, comprising:
a processor and memory, the memory comprising programmed instructions executable by the processor to:
receive a first input from a first sensor capable of recording swallowing and/or chewing activity;
receive a second input from a second sensor indicating a glycemic response;
determine an amount of food consumed based on the first input;
determine a type of food based on the second input; and
generate a number of calories consumed at least in part based on an amount and the type of food.

17. The system of claim 16, wherein the programmed instructions are further executable to send the number of calories to a database.

18. The system of claim 16, wherein the first sensor and/or the second sensor are incorporated into a wearable computing device.

19. The system of claim 16, wherein the first sensor comprises an accelerometer.

20. A system for tracking nutritional information about consumed food, comprising:
a processor and memory, the memory comprising programmed instructions executable by the processor to:
receive a first input from a first sensor capable of recording swallowing and/or chewing activity;
receive a second input from a second sensor indicating a glycemic response;
determine an amount of food consumed based on the first input;
determine a type of food based on the second input;
generate a number of calories consumed at least in part based on an amount and the type of food; and
send the number of calories to a database;
wherein the first sensor and/or the second sensor are incorporated into a wearable computing device.
